(19) United States
(12) Patent Application Publication
(10) Pub. No.: US 2017/0258518 A1
(43) Pub. Date: Sep. 14, 2017

(54) MULTI-TINE PROBE AND TREATMENT BY ACTIVATION OF OPPOSING TINES

(71) Applicant: LaZure Scientific, Inc., Issaquah, WA (US)

(72) Inventor: Larry AZURE, La Conner, WA (US)

(21) Appl. No.: 15/469,887

(22) Filed: Mar. 27, 2017

Related U.S. Application Data
(63) Continuation of application No. 12/210,956, filed on Sep. 15, 2008, now abandoned.
(60) Provisional application No. 60/972,705, filed on Sep. 14, 2007.

(51) Int. Cl. A61B 18/14; A61B 18/00

(52) U.S. Cl. CPC A61B 18/1477; A61B 18/14; A61B 2018/00267

(57) ABSTRACT

The present invention provides devices and systems, as well as methods, of electric field delivery and non-thermal or mild hyperthermia, and preferential or selective ablation of cancerous cells of target tissue regions. A method can include, for example, advancing a probe comprising a plurality of electrodes to a target tissue region comprising cancerous cells, and deploying the plurality of electrodes from a distal portion of a probe, and applying an alternating current so as to provide one or more electric fields extending through the volume and selectively or preferentially destroy cancerous cells within the volume.

This application claims the benefit under 35 U.S.C. §119(e) of U.S. Provisional Patent Application No. 60/972,705 (Attorney Docket No. 26533A-000900US), filed Sep. 14, 2007, the full disclosure of which is incorporated herein by reference.

### BACKGROUND OF THE INVENTION

The present invention relates generally to electric field delivery to tissue regions. More specifically, the present invention relates to electric field delivery and non-thermal or mild hyperthermic ablation of target tissue regions, including selective or preferential ablation of cancerous cells and solid tumors.

Current tissue ablation techniques often rely on a high-frequency, high temperature hyperthermia inducing electric current to the tissue of a patient (e.g., human, animal, etc.) as a means to remove unwanted tissue or lesions, staunch bleeding, or cut tissue. There has been increased interest and activity is the area of high temperature hyperthermal ablation as a tool to treat cancer by heat-induced killing and/or removal of tumor tissue.

In high-temperature hyperthermal tumor ablation techniques, high-frequency RF (e.g., “RF high-thermal ablation”) or microwave sources are used to heat tissue resulting in histological damage to the target tissue. In high-temperature RF thermal ablation techniques, for example, high frequencies, including about 500 kHz and greater, are used to cause ionic agitation and frictional heating to tissue surrounding a positioned electrode. Lethal damage to tissue (e.g., denaturation and cross-linking of tissue proteins) occurs at temperatures well in excess of about 47 degrees C., though heat generated near electrodes in RF thermal ablation can reach temperatures up to or exceeding about 100 degrees C.

A number of different cancer ablation methods and devices relying on high-temperature hyper-thermal ablation or high heat-induced tumor tissue destruction have been proposed. One such example includes U.S. Pat. No. 5,827,276, which teaches an apparatus for volumetric tissue ablation. The apparatus includes a probe having a plurality of wires journaled through a catheter with a proximal end connected to the active terminal of a generator and a distal end projecting from a distal end of the catheter. Teachings include a method and probe deployable in a percutaneous procedure that will produce a large volume of thermally ablated tissue with a single deployment.

U.S. Pat. No. 5,935,123 teaches a high-temperature RF treatment apparatus including a catheter with a catheter lumen. A removable needle electrode is positioned in the catheter lumen in a fixed relationship to the catheter. The treatment apparatuses are taught as being used to ablate a selected tissue mass, including but not limited to a tumor, or treat the mass by hyperthermia. Tumor sites are treated through hyperthermia or ablation, selectively through the controlled delivery of RF energy.

Numerous other methods and devices are taught using high-temperature hyper-thermal or high heat-induced cancer tissue destruction. However, a significant limitation of high-temperature RF induced, hyper-thermal ablation is the difficulty of localizing the heat-induced damage to targeted cancerous tissue while limiting histological damage and destruction to surrounding healthy, non-target tissue.

Thus, there is a need for minimally invasive ablation techniques that more preferentially or selectively destroy cancerous cells while minimizing damage to healthy tissue.

### BRIEF SUMMARY OF THE INVENTION

The present invention provides systems, devices and related methods for applying electric fields for cancerous cell destruction and ablation. Devices of the present invention will generally be designed to advance an electrode or plurality of electrodes to a target tissue region and apply an electric field to the target tissue region. The electrode or plurality thereof is typically positioned such that the applied electric field extends throughout the target tissue region, including, for example, where the electric field radiates outwardly and/or in a plurality of directions through the target tissue. Additionally, the energy applied to the target tissue region can be selected such that electrically generated heat is minimized and may include induction or delivery of controlled, mild hyperthermia, but where excessive or undesirable elevations in tissue temperature can be avoided. In particular embodiments, the applied electric field is generally a low-intensity and intermediate frequency alternating current field sufficient to provide low-power or non-thermal (e.g., mild hyperthermia) ablation of target cells. Thus, the present invention provides the additional advantage of providing minimally invasive, selective ablation or destruction of cancerous cells.

In one embodiment, the target tissue region includes a mass or solid portion of tissue. Typically, the target tissue region includes cancerous cells including, for example, a target tissue region including a solid tumor. The volume of the tissue to be subject to the inventive methods can vary, and will depend at least partially based on the size of the mass of cancerous cells. Peripheral dimensions of the target tissue region can be regular (e.g., spherical, oval, etc.), or can be irregular. The target tissue region can be identified and/or characterized using conventional imaging methods such as ultrasound, computed tomography (CT) scanning, X-ray imaging, nuclear imaging, magnetic resonance imaging (MRI), electromagnetic imaging, and the like. Additionally, various imaging systems can be used for locating and/or positioning of a device or electrodes of the invention within a patient's tissue or at or within a target tissue region.

As set forth above, the electrodes are positioned and an electric field (e.g., alternating current electric field) is applied. Ablation techniques according to the present invention can be accomplished in some embodiments without an excessive or undesirable increase in local tissue temperature and without substantial or sustained high-temperature (e.g., greater than at least 10 degrees C. above body temperature or greater than 48 degrees C. average tissue temperature) thermal effects of energy application being a primary means by which tissue ablation occurs. Typically, the applied electric field includes a low-intensity, intermediate frequency alternating current. In one embodiment, for example, the electric current provides a voltage field less than about 50 V/em. In another embodiment, the electrical current includes a frequency between about 50 kHz and about 300 kHz. The voltage field and/or the frequency of the applied current can be held constant during energy application or varied. In certain embodiments, electrode configuration and field application can take advantage of tumor physiology, including, e.g., orientation of dividing/proliferating cells within a target tissue region, and ensure that the electric field provided is substantially aligned with a division axis of a dividing cancerous cell.

For a fuller understanding of the nature and advantages of the present invention, reference should be made to the ensuing detailed description and accompanying drawings. Other aspects, objects and advantages of the invention will be apparent from the drawings and detailed description that follows.

### BRIEF DESCRIPTION OF THE DRAWINGS

FIG. 1 illustrates a device according to an embodiment of the present invention.

FIGS. 2A through 2C illustrate a device according to another embodiment of the present invention.

FIGS. 3A and 3B show a device having,an electrode configuration according to an embodiment of the present invention.

FIG. 4 illustrates an electrode arrangement according to an embodiment of the present invention.

FIGS. 5A and 5B illustrate a catheter and microcatheter device according to another embodiment of the present invention.

FIGS. 6A and 6B illustrate a method according to an embodiment of the present invention,

FIGS. 7A and 7B illustrate a method according to another embodiment of the present invention.

FIGS. 8A and 8B illustrate a tumor or mass of cancerous cells, with FIG. 8B showing a focused view of a dividing cancerous cell.

FIGS. 9A through 9D show a device and method according to an embodiment of the present invention.

FIG. 10 shows a device according to an embodiment of the present invention.

FIGS. 11A through 11C illustrates an ablation method according to an embodiment of the present invention.

FIGS. 12A through 12F illustrate exemplary electrodes according to various embodiments of the present invention.

FIGS. 13A and 13B illustrate a device according to an embodiment of the present invention.

FIGS. 14A and 14B illustrate a device according to another embodiment of the present invention.

FIGS. 15A through 15D illustrate a device and ablation method according to an embodiment of the present invention. FIGS. 15A and 15B show a cross-sectional, front view and side view, respectively, of a probe including microcatheters with deployable electrodes. FIGS. 15C and 15D show a first phase and a second phase of deployment, respectively.

FIG. 16 illustrates a system according to an embodiment of the present invention.

FIGS. 17A through 17D illustrate a probe according to an embodiment of the present invention, with deployment of guide tubes and associated electrodes, and field application with outer electrodes.

### DETAILED DESCRIPTION OF THE INVENTION

The present invention provides systems and devices, and related methods for tissue ablation. According to the present invention, an electrode or plurality of electrodes can be introduced into a target tissue region and an electric field applied to the target tissue region. The energy applied to the target tissue region can be selected such that electrically generated heat is minimized and, while may include induction or delivery of controlled, mild hyperthermia, excessive or undesirable rises in tissue temperature can be avoided, thereby providing low-power or non-thermal/mild hyperthermic ablation of target cells. Devices and methods of the present invention have been demonstrated to be effective in ablating cancerous cells without an excessive or undesirable thermal effect (e.g., average tissue temperature increases substantially above a 10 degree increase compared to body temperature, or substantially above about 48 degrees C. for substantial or prolonged periods) being a factor in the ablation process, with ablation occurring primarily among abnormally proliferating cells or cells exhibiting unregulated growth (e.g., cancerous cells). Thus, the present invention is advantageous in providing minimally invasive, selective ablation or destruction of cancerous cells, while leaving normal cells or tissue substantially intact.

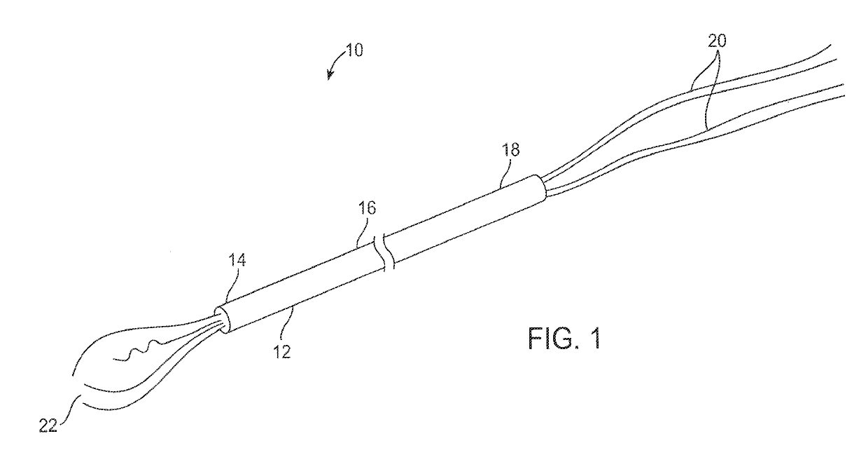

Referring to FIG. 1, a device according to an embodiment of the present invention is described. The device 10 includes a delivery member 12 having a distal portion 14 and a proximal portion 16. The device 10 further includes a proximal portion 18 of the device that can he coupled (e.g., removably coupled) to the delivery member 12. Additionally, the device 10 can include conductive cables 20 electrically coupled to an energy source (not shown). The device includes a plurality of electrodes 22 at the distal portion 14 of the delivery member 12. The electrodes 22 can be positioned or fixed, for example, at the distal end of the delivery member 12 or positionable and deployable from a lumen of the delivery member 12 and retractable in and out of the distal end of the delivery member 12. The electrodes 22 can include a non-deployed state, where the electrodes 22 can be positioned within a lumen of the delivery member 12, and a deployed state when advanced from the distal end of the delivery member 12. Electrodes 22 are advanced out the distal end and distended into a deployed state substantially defining an ablation volume.

The present invention can include a variety of electrode compositions, configurations, geometries, etc. In certain embodiments, electrodes can include tissue-penetrating electrodes including, for example, small diameter metal wires having tissue-piercing or sharpened distal ends that can penetrate tissue as they are advanced within the target tissue region. Electrodes can be non-insulated or can include an insulated portion. In one embodiment, a non-insulated portion of the electrode provides an electric field delivery surface for delivery of electrical current to the surrounding tissue. Electrodes can be substantially rigid, e.g., so as to be more easily advanced through tissue, including hardened or more dense tissue, or can be more flexible, depending upon the desired use. In one embodiment, an electrode includes a needle or needle-like electrode or electrode having a substantially linear portion. In another embodiment, electrodes can be curved, having a curved portion or portion with a radius of curvature. Electrode composition can vary and in certain embodiments can include a memory metal (e.g., commercially available memory metals, Nitinol™, etc.) or sprung steel. Suitable electrode materials can include, e.g., stainless steel, platinum, gold, silver, copper arid other electrically conductive materials, metals, polymers, etc. In certain embodiments, electrodes can be positioned in and deployable from a lumen of a catheter and/or microcatheter or other member for introducing the electrode into a tissue.

In another embodiment, the present invention can make use of one or more sensor mechanisms to provide feedback and/or control the ablation process. Sensor mechanisms can include sensors or detectors that detect and measure parameters such as temperature, current, voltage, impedance, pH and the like. Certain embodiments of the present invention can include modifying the applied electric power or current at least partially based on a detected characteristic or a change in a detected characteristic. In one embodiment, for example, modification of the applied electric power or current can occur in response to a measured temperature, impedance, and the like. Modification can include, for example, modifying the voltage, frequency, etc. of the applied current and/or discontinuing application of the electric current, for example, where the ablation process or a stage thereof is determined to be completed.

A target tissue region can be located anywhere in the body where the tissue ablation methods of the present invention would be desired or beneficial. Target tissue is not limited to any particular type and non-limiting examples can include, e.g., breast tissue, prostate tissue, liver, lung, brain tissue, muscle, lymphatic, pancreatic tissue, colon, rectum, bronchus, and the like. The target tissue region will typically include a mass or solid portion of tissue. Typically, the target tissue region includes cancerous cells including, for example, a target tissue region including a solid tumor, and may include a volume of tissue including both cancerous and non-cancerous cells (e.g., mixed population of cells). The term “cancerous cell”, as used herein, generally refers to any cells that exhibit, or are predisposed to exhibiting, unregulated growth, including, for example, a neoplastic cell such as a premalignant cell or a cancer cell (e.g., carcinoma cell or sarcoma cell), and are amenable to the ablation methods described herein. The volume of the tissue to be subject to the inventive methods can vary depending, for example, on the size and/or shape of the mass of cancerous cells, as well as other factors. Peripheral dimensions of the target tissue region can be regular (e.g., spherical, oval, etc.), or can be irregular.

Imaging systems and devices can be included in the methods and systems of the present invention. For example, the target tissue region can be identified and/or characterized using conventional imaging methods such as ultrasound, computed tomography (CT) scanning, X-ray imaging, nuclear imaging, magnetic resonance imaging (MRI), electromagnetic imaging, and the like. In some embodiments, characteristics of the tumor, including those identified using imaging methods, can also be used in selecting ablation parameters, such as energy application as well as the shape and/or geometry of the electrodes. Additionally, these or other known imaging systems can be used for positioning and placement of the devices and/or electrodes in a patient's tissues.

As set forth above, the electrode is positioned within the target tissue region and the applied electric field is sufficient to provide low-power or non-thermal/mild hyperthermic ablation of target cells. The term “non-thermal ablation” as used herein generally refers to techniques of the present invention including the removal of or destruction of the function of tissue or cells of a tissue by application of an electric field, and where the energy application/delivery process occurs without a substantial increase in local tissue temperature above or beyond mild temperature increases due to mild or low-level hyperthermia, and without high-temperature thermal effects (e.g., substantially above 10 degree increase in average tissue temperature in the target region) of energy application being a significant or primary means by which tissue ablation occurs. In some embodiments, a substantial increase in local tissue temperature can be avoided altogether, with no resulting apparent increase in temperature being detected in the target tissue region. In some embodiments, however, small changes/elevations in temperature in the target tissue region may occur, but will typically be no more than a few degrees C. above body temperature (e.g., less than about 10 degrees C., but typically no more than about 2 degrees above body temperature), and without the high-temperature thermal effects (e.g., average tissue temperature above about 48-50 degrees C.) being the primary means by which tissue ablation occurs (e.g., no significant thermally-mediated, lethal protein denaturation and cross-linking). In some instances, energy delivery can be selected so as to deliver or establish low-level or mild increases in average tissue temperature of the target tissue/region, including delivery of mild hyperthermia to the tissue. As described above, mild hyperthermia may include an increase of the average tissue temperature up to about 10 degrees C. above body temperature (e.g., normal human body temperature of about 38 degrees C.). Thus, mild hyperthermia can include increased temperature up to about 48 degrees C., but will typically be controlled to prevent average tissue temperatures exceeding 50 degrees C. Target temperature ranges for energy delivery and resulting mild hyperthermia induction, according to the present invention, generally range from about 40-47 degrees C., and more typically about 42-45 degrees C. As target tissue temperatures rise above about 40-42 degrees C., the cytotoxic effects of energy delivery on cancerous cells of the target region is observably enhanced, possibly due to an additive and/or synergistic effect of current field and hyperthermic effects. Where hyperthermic effects are substantially maintained below about 48 degrees C., the energy delivery according to the present invention appears to more preferentially destroy cancerous cells compared to healthy or non-cancerous cells of the target tissue region. Where energy delivery induces tissue heating substantially in excess of about 45-48 degrees C., the preferential cytotoxic effects on cancerous cells begins to diminish, with more indiscriminate destruction of cancerous and non-cancerous cells occurring. Thus, a significant advantage of treatment methods according to the present invention includes the ability to precisely and accurately control energy delivery and induced hyperthermic effects, such that tissue hyperthermia can be accurately controlled and maintained in a desired temperature range(s) e.g., temperature ranges selected for more targeted or preferential destruction of cancerous cells compared to non-cancerous cells.

Typically, the applied electric field includes a low-intensity, intermediate frequency alternating current. The intermediate frequency employed according to the present invention, for example, will be less than that typically required for frictional/resistance heating to tissue surrounding the electrode (e.g., less than about 400 kHz, preferably about 300 kHz or less) In one embodiment, for example, the electric current provides a voltage field less than about 50 V/cm. In another embodiment, the electrical current includes a frequency between about 50 kHz and about 300 kHz.

The voltage field and/or the frequency and/or magnitude of the applied current can be held constant during energy application or varied. One or more treatment phases can be applied, with each phase having selected treatment parameters (e.g., energy parameters, duration, etc.). In some embodiments, providing a non-constant or varying voltage and/or frequency and/or current by “scanning” across a given range may be desired, for example, to ensure that the optimal ablative voltage/frequency/current is applied to the target tissue region. In another embodiment, a particular voltage/frequency/current can be selected prior to energy application. In yet another embodiment, the voltage field can be turned “on” and “off” at a frequency high enough to keep the temperature of the tissue relatively constant, and varying the on/off duty cycle (e.g., “on” time vs. “off” time) to more precisely control the temperature of the target tissue. Furthermore, the electrode(s) can be positioned within the target tissue region such that electrical current application occurs from within the target tissue, and the target tissue is ablated from the inside out. In one embodiment, electrode(s) are positioned within the target tissue region (e.g., tumor) and the applied electrical current provides an electric field extending radially outward from the electrode. In certain embodiments, such positioning can take advantage of tumor physiology, including, e.g., orientation of dividing/proliferating cells within a target tissue region, and ensure that the electric field provided by the electrode is substantially aligned with a division axis of a dividing cancerous cell, or otherwise established through a tissue volume in a plurality of directions.

Particular energy application or treatment times can be selected according to the present invention. Continuous treatment times have been administered in both longer time increments (e.g., about 12 hours) and shorter increments of a few hours or less (e.g., treatment times of about 1.5 to about 3 hours, to less than 30 minutes). In most instances, significant cancerous cell destruction was observed within 90 minutes, and in some cases, tumors were virtually undetectable after less then 30 minutes of treatment. Thus, in certain embodiments a particular treatment phase will include energy application of less than 12 hours, and more typicially less than 3 hours. In many instances, a phase of treatment can include a less than 30 minute energy application. Since indications are that energy delivery as described herein can be safely administered for longer periods of time, longer treatment times can be included if necessary (e.g., several days of continuous treatment). Additionally, various treatment phases or “doses” can be administered to a patient over a period of time (e.g., days, weeks, months, longer) and can include multiple phases of treatment for the same tissue region or tumor, or can address different tissue regions or tumors (e.g., secondary tumors, etc.).

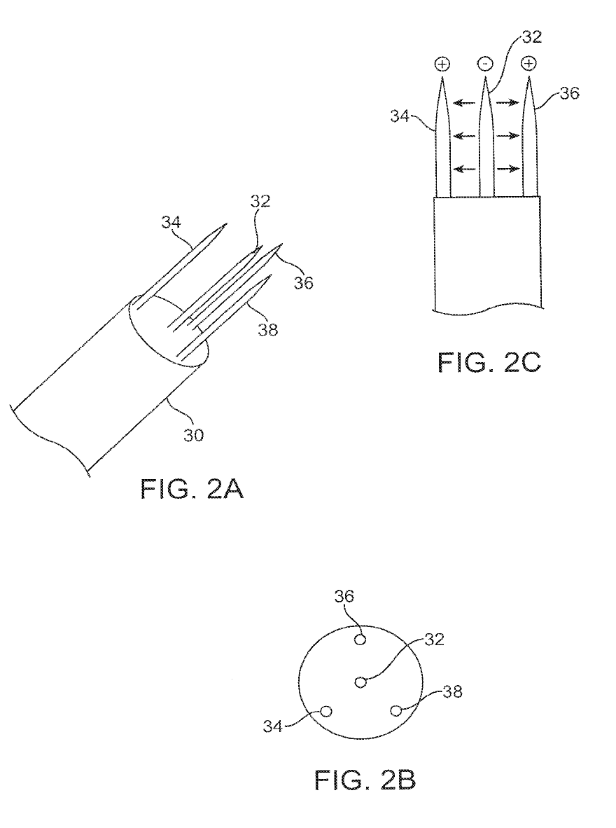

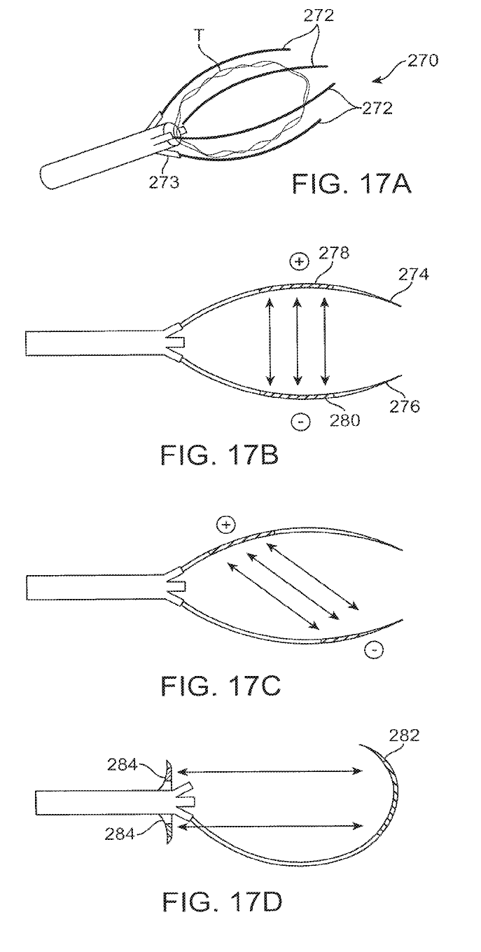

FIGS. 2A through 2C show a device having a plurality of electrodes according to another embodiment of the present invention. As shown, the device 30 includes a plurality of electrodes extending from the distal portion of the device. FIG. 2A shows a three dimensional side view of the device having the plurality of electrodes. FIG. 2B shows a top view of the device illustrating the electrode arrangement. The plurality includes a centrally positioned electrode 32 and outer electrodes 34, 36, 38 spaced laterally from the central electrode 32. The illustrated electrodes include substantially linear needle-like portions or needle electrodes. The electrodes extend from the distal portion of the device and are oriented to be substantially parallel with the longitudinal axis of the device 30. Additionally, each electrode is substantially parallel with other electrodes of the plurality. The plurality of electrodes substantially define the ablation volume, with the outer electrodes 34, 36, 38 substantially defining a periphery of the ablation volume and the electrode 32 positioned within or at about the center point of the defined periphery. Each of the electrodes can play different roles in the ablation process. For example, there can be changes in polarity and/or polarity shifting between the different electrodes of the device. As with other devices of the invention, electrodes can be electrically independent and separately addressable electrically, or two or more electrodes can be electrically connected, for example, to effectively function as one unit. In one embodiment, for example, outer electrodes 34, 36, 38 can be electrically connected and, in operation, include a polarity different from that of the inner electrode 32. As illustrated in FIG. 2C the electrodes 32 and 34, 36 of the device can include opposing charges (e.g., bipolar). In such an instance, the applied electrical current can provide an electrical field, as illustrated by the arrows, extending radially outward from the central electrode 32 and toward the peripherally positioned or outer electrode(s) 34, 36. Electrodes of a plurality (e.g., as illustrated in FIG. 2A-2C and elsewhere) can be activated in groups or pairs for establishing different current fields or field orientations through the target tissue. As described further herein (see, e.g., FIGS. 17A-17D), different pairs of electrodes of a device or probe of the present invention can be differentially activated (e.g., in seriatim) so as to establish different current fields or field directions/orientations through a target tissue.

In some embodiments, devices and/or systems of the present invention include electrically floating systems or systems designed to operate without an earth grounding. In some instances, it was observed that electrode configurations that were electrically floating in this manner allowed more accurate or controllable field application and/or delivery. The low-power requirements of systems according to certain embodiments allow more design options in configuring devices and systems that are electrically floating, as described, compared, for example, to known techniques such as thermal RF or microwave ablation, or high-voltage irreversible electroporation that require much higher powered energy delivery and corresponding power sources.

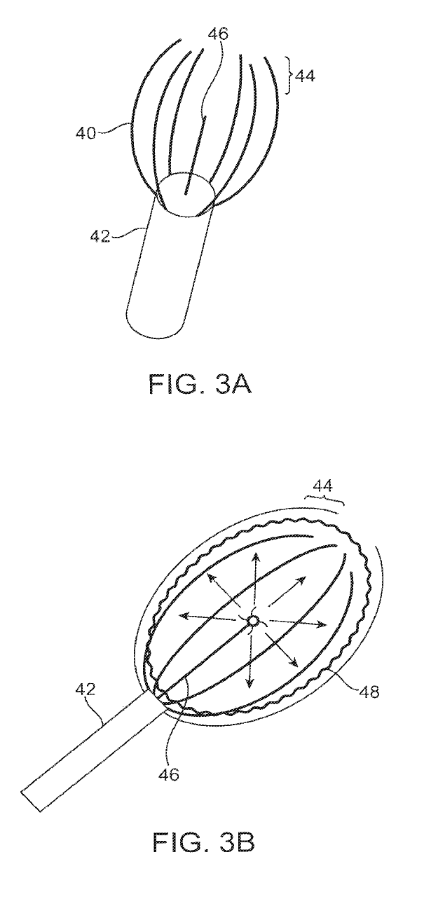

Another embodiment of a device of the invention is described with reference to FIGS. 3A and 3B. The device 40 includes a plurality of electrodes at or extending from the distal end 42 of the device 40. The plurality of electrodes includes outer positioned electrodes 44 that are curved and substantially define an ablation volume. An electrode 46 is positioned within the volume defined by the outer electrodes 44 and spaced from the electrodes 44. The central electrode 46 is shown as being substantially linear and parallel with the longitudinal axis of the device 40, although other configurations will be available. FIG. 3B shows a target tissue 48 within the periphery defined by the outer electrodes 44 with an electrical current being applied to the target tissue 48, and illustrating an oblong or oval ablation volume being defined by the curved electrodes 44. Thus, a target tissue region 48, such as a solid tumor, can essentially be encased within the volume defined by the outer electrodes 44. Arrows illustrate an electric field extending outward and radially from the electrode 46 and in a plurality of different directions.

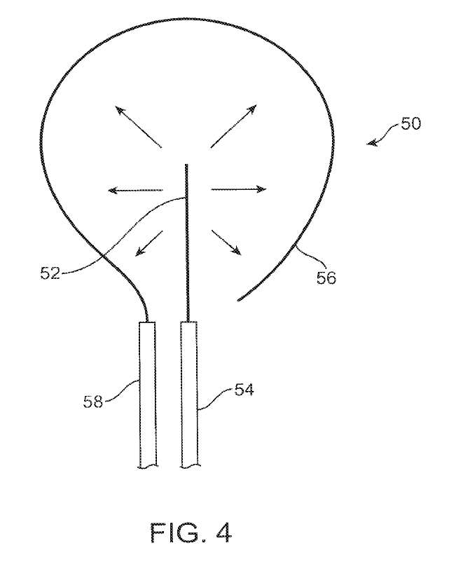

Electrodes of a device according to another embodiment of the present invention are described with reference to FIG. 4. The device 50 includes a substantially linear electrode 52 that is retractable in and out of a microcatheter 54 and an electrode 56 having a curved portion, the electrode retractable in and out of a microcatheter 58. Microcatheters 58 and 54 can be included in a single delivery member, such as in a lumen(s) of a delivery catheter or can be independently arranged, e.g., for individually accessing and addressing a target tissue. One outer electrode is illustrated (e.g., electrode 56), though multiple outer or secondary electrodes can be provided, as illustrated in other embodiments (e.g., see below).

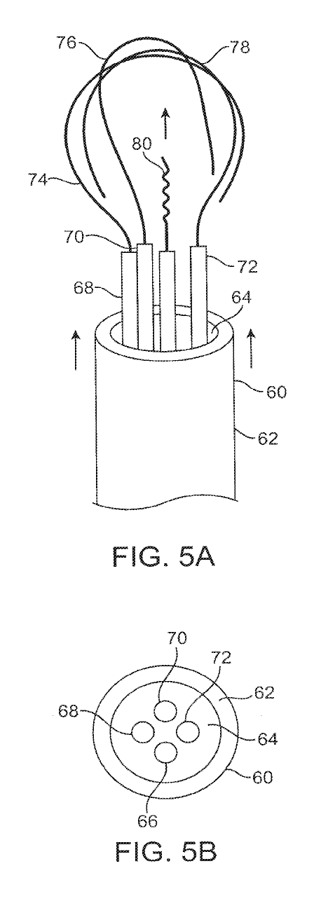

A device can include a plurality of electrodes, each deployable or retractable in and out of a microcatheter, with each microcatheter/electrode assembly optionally positioned within a central lumen of a larger delivery member, as illustrated in FIGS. 5A and 5B. The device 60 includes a delivery member 62 with a lumen 64, and microcatheters 66, 68, 70, 72 positioned in the lumen. FIG. 5B shows a top view of the device with microcatheters 60, 68, 70, 72 positioned in the lumen 62 of the delivery member 60. Electrodes 74, 76, 78 each having a curved portion, are deployable from microcatheters 68, 70, 72 and, in a deployed state, substantially define an ablation volume. Electrode 80 is deployable from microcatheter 66 is positioned within the ablation volume substantially defined by electrodes 74, 76, 78.

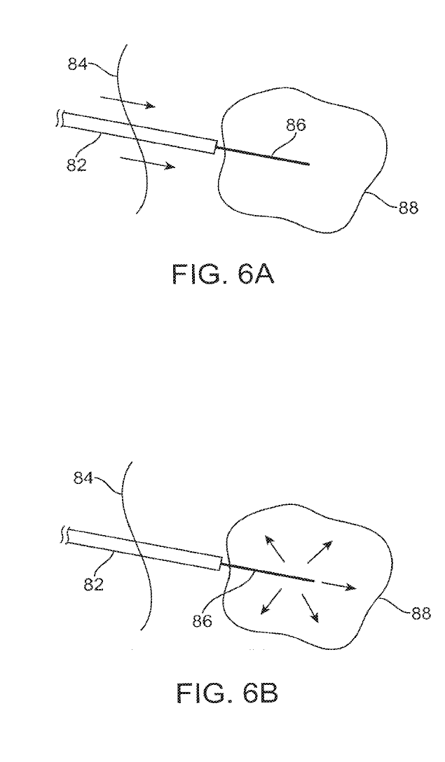

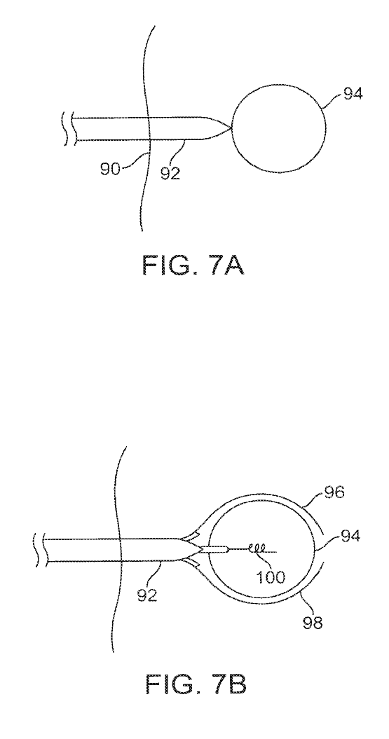

In use, as shown in FIG. 6, a device 82 of the present invention can be advanced through the patient's tissue 84, and an electrode 86 of the device 82 positioned within a target tissue region 88 (e.g., tumor). Once the electrode is positioned in the target tissue region 88, electrical current is delivered to the target tissue region 88. As the electrode 86 is positioned within the target tissue region 88, the applied electrical current can provide an electric field that radiates outward and in a plurality of directions. A system or device of the invention can be operated in monopolar mode or bipolar mode. In one monopolar operation embodiment, a second electrode can be placed, for example, outside the patient's body, such as by positioning the patient on a conductive pad or plate (e.g., metal plate) and may make use of conductive materials, such as conductive gels or adhesives, placed between the patient's skin and the second electrode. In a bipolar mode embodiment, outer electrodes substantially defining an ablation volume can function as return electrodes, or complete a circuit with an electrode(s) positioned within the ablation volume, with applied current flowing through tissue of the target region positioned between the outer electrodes and electrode(s) positioned within the ablation volume. FIG. 7 shows use of a device of the present invention according to another embodiment of the present invention. As described above, the device 90 is advanced through the patient's tissue and the delivery member 92 positioned proximate to the target tissue region 94. Once the delivery member 92 is positioned, a plurality of electrodes 96, 98, 100 can be deployed from the delivery member 92. Outer electrodes 96, 98 are deployed within or around the perimeter of the target tissue region 94, e.g., at about the margin of the target tissue region (e.g., tumor margin) and substantially define the ablation volume or target region. The inner electrode 100 is positioned within the ablation volume.

The present invention can include various means of accessing or addressing a target tissue and positioning electrodes/probes for delivery of the described ablative treatment. Typically, positioning of a device of the invention will include a minimally invasive access and positioning techniques, including, e.g., access techniques commonly used with other types of tissue ablation (e.g., thermal RE ablation, microwave ablation, high-voltage electroporation, and the like). For example, devices of the invention can be introduced percutaneously through the skin and advanced through tissue and positioned at a target tissue. Though, addressing a target tissue and positioning of a device can occur in conjunction with more conventional surgical techniques or laparoscopic techniques.

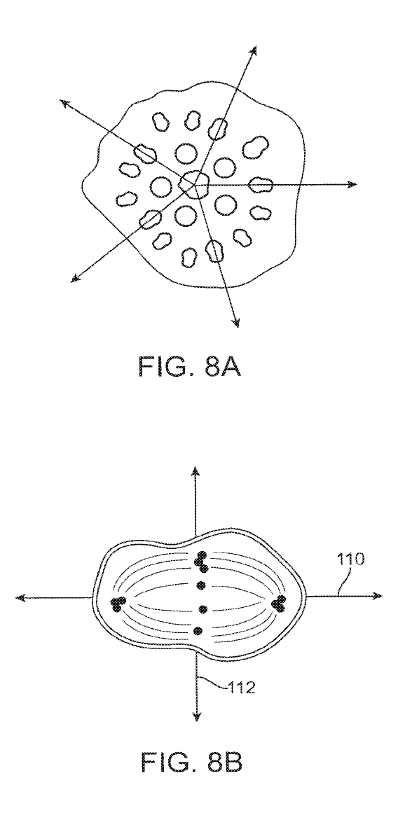

As set forth above, certain embodiments of the present invention include positioning of an electrode within the target tissue region and applying an alternating electrical current, with the applied electrical current providing an electrical field that radiates outwardly from the positioned electrode. Electric field application in this manner was found to be highly effective in disrupting and destroying cancerous cells via low-power ablation and in the absence of a sustained high-temperature, thermal ablative effect (e.g., substantially in excess of 48 degrees C.). In certain embodiments, disruption of cancerous cells and resulting ablation according to the present invention effectively occurred where the electrical field provided by an electrode of an inventive device was applied in a radial field orientation, with fields presumably, based on tumor physiology, more substantially aligned with a division axis of a dividing cancerous cell or plurality of cells. FIG. 8A shows a simplified version of a growth pattern and physiology of a cancer tumor or solid mass of cancerous cells, illustrating tumor growth by cancer cells dividing outwardly from the center of a region. Arrows indicate division axes of cancerous cells dividing outwardly from the center. FIG. 8B shows a focused and simplified view of a dividing cell of the tumor of FIG. 8A, further illustrating the concept of an axis of cell division. The illustrated dividing or proliferating cancerous cell (illustrated at a metaphase stage of mitosis) includes an axis of cell division 110 substantially orthogonal to a metaphase plate axis 112, where the cell divides substantially along the plate axis 112 and cell proliferation and growth occurs along the cell division axis 110. Thus, in certain embodiments of the invention, the positioning of an electrode within a tissue region, e.g., proximate to the center region of a tumor or mass of cancer cells, and/or the configuration and arrangement of the electrodes of the device, can be selected such that the electrical field radiates outwardly from about the center region and the electric field is substantially aligned with the division axes of cells of the growing tumor (e.g., based on tumor physiology), or across a tissue volume having a mixed population of cancerous and healthy cells.

Furthermore, the electric field application as described was observed to be particularly effective in selectively disrupting and destroying the dividing cancerous cells, while having little or no effect on normal cells that were not exhibiting unregulated growth and proliferation. Without being hound by any particular theory, electric field application as described may specifically disrupt the cell division process (e.g., mitosis) or progression through the cell cycle, or a stage or process thereof (e.g., mitotic spindle formation, microtubule polymerization, cytoplasmatic organelle function or arrangement, cytokinesis, cellular osmotic balance or the like) and, therefore, more particularly effects cells exhibiting unregulated growth (e.g., cancerous cells) and progressing more rapidly through the cell cycle.

According to the present invention, a target tissue region can be ablated in whole or in part. It will be recognized that while it is generally desirable to ablate as much of the target region or tumor as possible, in some embodiments, methods can include ablation of a portion or less than the entirety of the target region. In some instances, partial tumor ablation can be sufficient to ultimately destroy or kill an entire tumor or cancerous tissue region.

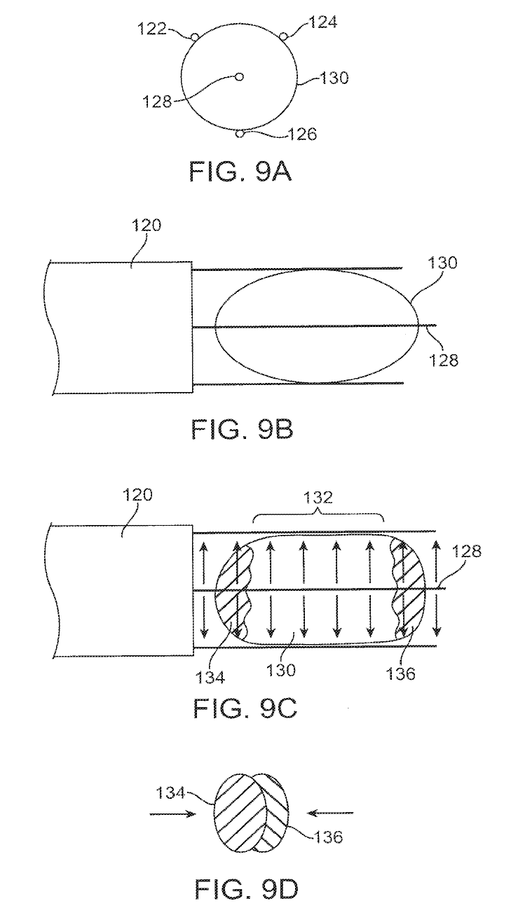

Use of a device according to an embodiment of the invention (e.g., the device of FIG. 2A through 2C) is discussed with reference to FIGS. 9A through 9D. The device 120 includes a plurality of electrodes, including outer electrodes 122, 124, 126 substantially defining an ablation volume and at least one inner electrode 128. The device can be positioned at a target tissue region including a tumor or portion thereof. The tumor 130 is shown positioned substantially within the ablation volume, with the inner electrode 128 positioned about through the center of the tumor and outer electrodes 122, 124, 126 spaced laterally from the inner electrode 128 and positioned at about the tumor margin, or slightly inside or outside the tumor margin. FIG. 9A shows a top sectional view of the tumor 130 and positioned electrodes 122, 124, 126, 128, and FIG. 9B shows a side view of the same. An electric field, illustrated by the arrows in FIG. 9C, is provided by the positioned electrodes and the application of an electrical current. As can be seen, in the parallel straight needle electrode configuration shown in FIGS. 9A through 9C, the electrical field along the length of the ablation volume is oriented in a direction orthogonal to the longitudinal axis of the device. The electric current emanating from the center electrode 128 toward the outer electrodes 122, 124, 126 provides a field that is substantially aligned with the direction of cell division for many of the tumor cells, particularly those in region 132, which divide in a direction from the tumor center and outward (see, e.g., FIGS. 8A and 8B). It will be recognized that arrows are provided for illustrative purposes, and that embodiments of the invention are not limited to any particular current and/or electrical field direction, but may include directions other than and/or in addition to those specifically illustrated. The tumor includes region 132 where direction of tumor cell division is believed more closely aligned with the electrical field. In the illustrated configuration, the tumor can include regions 134, 136 at opposing ends of the tumor that may include a greater proportion of cells having cell division axes not in alignment with the provided electric field, or, in other words, are at an angle relative to the electric field and may remain alive following application of energy, while a greater proportion of cells of region 132 are ablated. However, in one example, using tumor ablation in this manner, the tissue/cells of region 132 were ablated and materials subsequently removed from the treatment site (e.g., squeezed out by application of pressure) acid/or absorbed by surrounding tissue, and regions 134 and 136 were observed to collapse inward forming a flat, “pancake-like” tissue residue (FIG. 9D), which eventually died subsequent to energy application. Remarkably, numerous experimental (e.g., animal) models that were subject to the described ablation techniques of the present invention demonstrated complete remission of detectable tumor. These results indicated that the present inventive methods effectively ablate tumor tissue, can destroy a solid tumor, even where less than the entirety of tumor tissue is ablated, and illustrated the improved tissue ablation where electric field may he aligned with the direction of cell division of cancerous cells, based on tumor physiology.

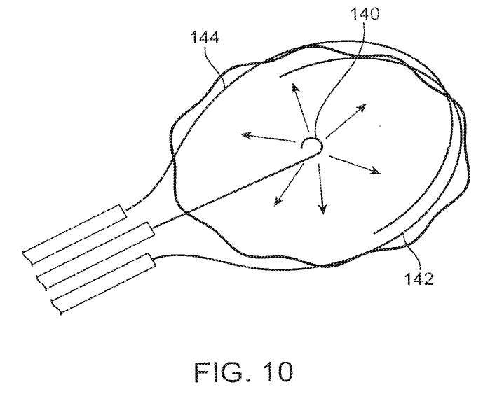

Another embodiment of a device of the present invention is illustrated in FIG. 10. As discussed above, device configuration and electrode arrangement can be selected such that the electrical field radiates outwardly from about the center of the target tissue region and the electric field is substantially aligned with the division axes of certain cells of the growing tumor. More optimal application of electrical energy and alignment of the electric field with division axes of the growing tumor can be accomplished by both positioning of the electrodes in the target region and selected electrode configuration and/or geometry of the device. In one embodiment, for example, device can include an inner electrode 140 and a plurality of outer electrodes 142, 144 that are curved. The inner electrode 140 can additionally include a curved or non-linear distal portion. Having curvature on electrodes can help select an applied electric field that radiates in a plurality of directions, including directions other than orthogonal to the longitudinal axis of the device or inner electrode. The outer curved electrodes substantially define the ablation volume and the inner electrode is positioned within the ablation volume. Arrows illustrate the field emanating from the center in a plurality of directions and substantially in line with dividing cancerous cells of the target tissue region. In some instances, the electric field provided by this configuration may align with a greater portion of cancerous cells of the target tissue region compared, for example, to the straight needle electrode configuration illustrated in FIGS. 9A through 9D.

As the ablation process is initiated, the field intensity is highest at the inner or central electrode and within tissue around and in close proximity to the inner or central electrode. As the ablation process progresses, cancerous cells near the inner electrode are observed to be destroyed or ablated first. The ablated cells effectively “liquefy” or assume properties of a low impedance, liquid-like material. The term “liquefy” is used herein for convenience and illustrative purposes, and does not necessarily imply any particular mechanism of ablation or cell death, which may include cell blebbing, apoptosis, lysis, or some other cellular process, and/or some combination thereof. Another possible cause of cell destruction may include disruption of cellular membrane integrity, e.g., including dielectric breakdown of one or more cellular membranes (see, e.g., below). The liquid-like material surrounds the central electrode and effectively enlarges the higher field intensity ablative area, with the highest field intensity ablative area being at the outer perimeter of the liquid-like material. Thus, the liquid-like material is said to become a “virtual electrode”. As the ablation process progresses, the outer perimeter of the liquid-like material or “virtual electrode” expands, essentially ablating the target tissue region from the inside out. In some embodiments, target tissue regions were observed to be more pliable and soft or mushy following the ablation process. The ablated, liquid-like tumor tissue was eventually removed from the treatment site and/or absorbed by the surrounding tissue, and no longer detectible.

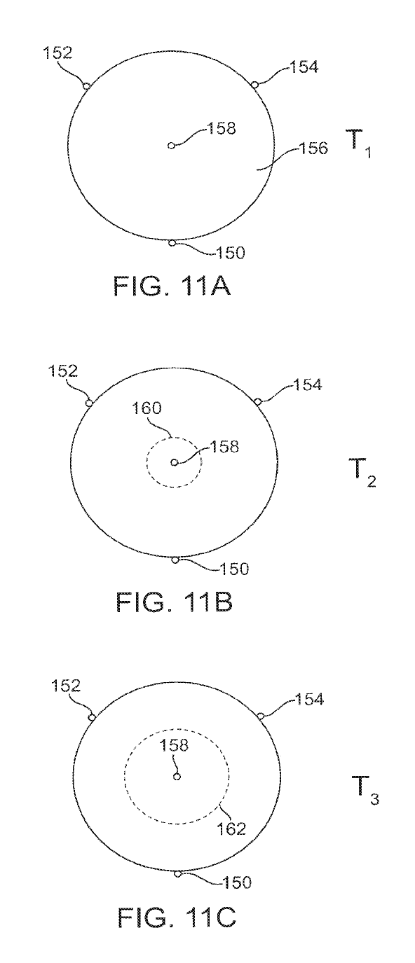

The virtual electrode effect is illustrated with reference to FIGS. 11A through 11C, showing a cross section view of electrodes positioned in a target tissue region. Outer electrodes 150, 152, 154 are positioned at about the margin or outer periphery of the tumor 156, and inner electrode 158 is positioned at about a center point of the volume defined by the outer electrodes 150. 152, 154. Ablation is shown at T1, or the beginning of the ablation process (FIG. 11A); T2 after ablation has begun with the expanding liquid-like tissue region 160 (FIG. 11B); and subsequent time T3, with the liquid-like tissue region 162 expanded further outward from the inner electrode 158 and toward the outer electrodes 150, 152, 154 (FIG. 11C).

The ablation process, including the progress thereof, can be monitored by detecting the associated change in impedance in the ablated tissue. Once the outer perimeter of the ablated, liquid-like tissue reaches the outer electrodes defining the ablation volume, the impedance stabilizes or levels out. Thus, the progress of the ablation process can be monitored by measuring change in impedance, and electric field application discontinued once a change in impedance is no longer observed.

Feedback measurements can also be used to ensure that the ablation of the target cancerous cells occurs by non-thermal or mild hyperthermal ablation, with average tissue temperatures maintained within a desired range or not reaching or exceeding undesirable tissue temperatures (e.g., in excess of 48-50 degrees C.) for sustained periods. In certain embodiments it may be desirable to generate as much field intensity at the inner electrode as possible without causing a hyper-thermal effect or thermal ablation. Certain hyper-thermal effects would he observable and distinguishable from the desired non-thermal ablation of the present invention, since thermal ablation would cause destruction of the surrounding cells without the “liquefying” effect described above. For example, if cell destruction is caused by a thermal ablation process, the impedance of the treated tissue may not decrease since the impedance of cells that are charred or become necrotic due to thermal effects typically increases. In one embodiment, non-thermal ablation according to the present invention can include placement of a sensor, such as a thermocouple, within the target tissue region (e,g., proximate to the inner electrode), and selection of an applied field intensity as below the intensity that would cause thermal effects on the target cells.

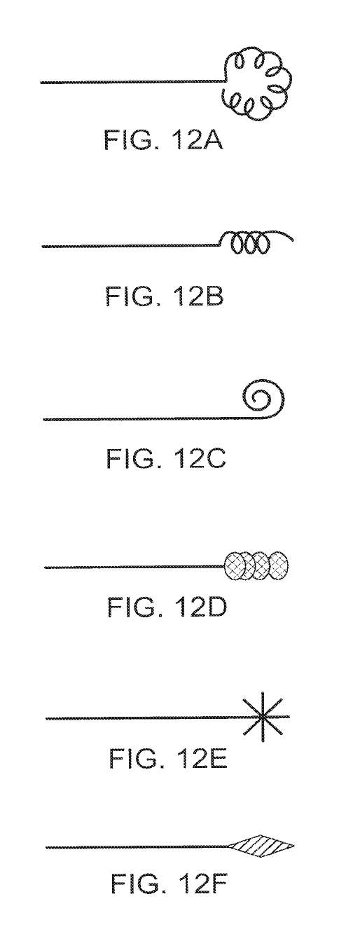

As stated above, in some instances, it may be desirable to increase the field intensity emanating from the position of the inner electrode within the target tissue region. In one embodiment of the present invention, field intensity can be increased by increasing the surface area of the inner electrode that is placed within the target tissue region. Various embodiments of increased surface area electrodes are illustrated in FIGS. 12A through 12F, though other configurations will be available. In one embodiment, the electrode includes a coiled distal portion that can further form a circular pattern (FIG. 12A), a corkscrew (FIG. 12B), or a simple coil (FIG. 12C). In another embodiment, a small wire mesh could be included at the electrode distal end, and expanded when placed within a target tissue region (FIG. 12D). In other embodiment, an electrode can include a “Litz” wire-type of electrode, where the distal end includes a plurality of small wires expanded in an array (FIG. 12E). In another embodiment, the distal portion can include a shape resembling two cones stacked base to base, or from a side view having a diamond shape (FIG. 12F). The pointed opposing distal and proximal portions of the double cone/diamond end can facilitate insertion and retraction of the electrode in tissue. Numerous other configurations are available and can include, for example, a ring, sphere, corkscrew, helix, concentric helixes, or plurality thereof, array of needles, length of non-resilient, string-like wire that is pushed out a tube and forms a small ball of wire similar to a string piling up randomly in a small container, and the like.

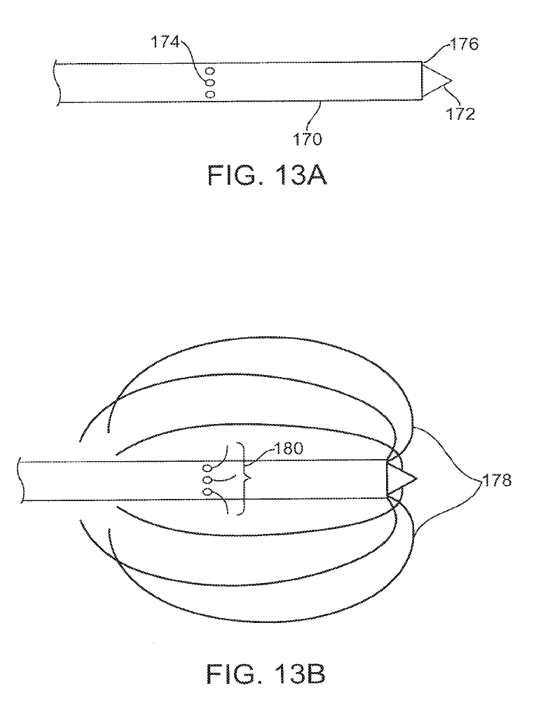

Another embodiment of a device of the present invention is shown in FIG. 13. The device includes a delivery member 170 with a tissue piercing distal portion 172. The delivery member includes a lumen and openings 174 on the body and at 176 the distal end. A plurality of electrodes are positionable within the lumen of the member. In a deployed state, outer electrodes 178 extend out the openings 176 at the distal end of the member 170 and invert in an umbrella-like orientation. The deployed outer electrodes 178 substantially define an ablation volume. Electrodes 180 extending out the openings 174 of the body are spaced from the outer electrodes 178 and positioned within the ablation volume.

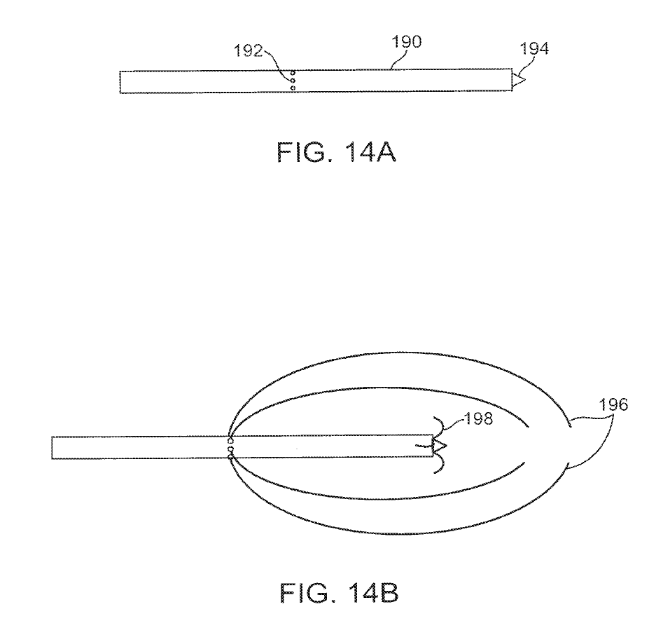

FIG. 14 illustrates a device similar to that shown in FIG. 13. Referring to FIG. 14, the device includes a delivery member 190 with a distal portion, openings 192 on the body and at the distal end 194. Outer electrodes 196 deploy distally out the body openings 192 and define a volume surrounding the electrodes 198 deployed and extending out the distal end opening 194.

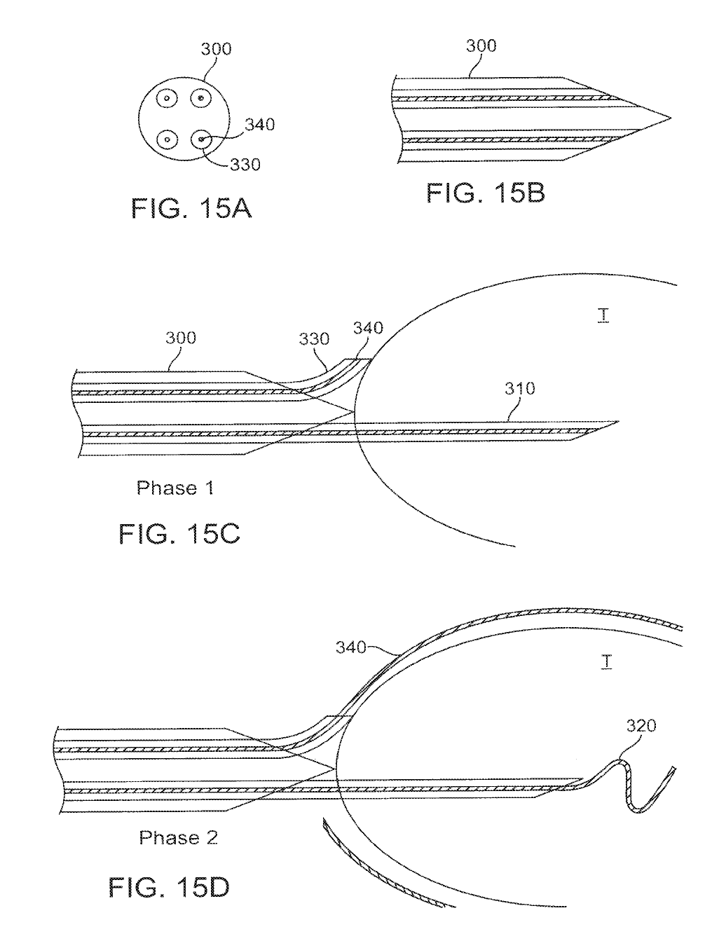

Another embodiment of a device of the invention is described with reference to FIGS. 15A through 15D. The device includes a plurality of electrodes positioned in a lumen of a delivery member 300 of a probe or delivery catheter, with each electrode positioned within a microcatheter as illustrated by microcatheter 330 and electrode 340, and each microcatheter positioned within the lumen of a delivery member 300. Microcatheters can act as guide tubes as advanced or deployed from delivery member 300 for initial aiming and/or positioning of electrodes contained therein (see below). FIG. 15A shows a cross-sectional front view of microcatheters positioned in the lumen of delivery member 300. The delivery member or probe 300 can include a tissue piercing end that is pointed or sharpened so as to more easily be inserted into the tissue of a patient, as illustrated in FIG. 15B. Similarly, a microcatheter (e.g., microcatheters 310, 330) can include a pointed or sharpened tissue piercing end. In use, the delivery member 300 can be advanced through the tissue of a patient and the distal end positioned proximate to a target tissue region (e.g., tumor “T”) and the microcatheters are deployed from the delivery member for positioning of electrodes in a desired arrangement. As shown in phase I deployment (FIG. 15C), microcatheter 310 is advanced distally from the distal end of the delivery member and into the target tissue region, where the electrode 320 of the microcatheter can be deployed. Microcatheters can include shape memory metal (e.g., Nitinol) such that microcatheters assume a desired and/or predetermined shape when deployed from the delivery member 300, as illustrated with microcatheter 330. Thus, microcatheter 330 can also be deployed from the delivery member 300 to aim the electrode 340. In phase 2 deployment (FIG. 15D), electrode 340 is deployed in the direction aimed by microcatheter 330, such as around the outer perimeter of the target tissue region (e.g., tumor margin). Both microcatheters and electrodes positionable therein can be made of memory shape metal such as nitinol so as to assume a predetermined configuration when deployed. Other phases of use can further be included.

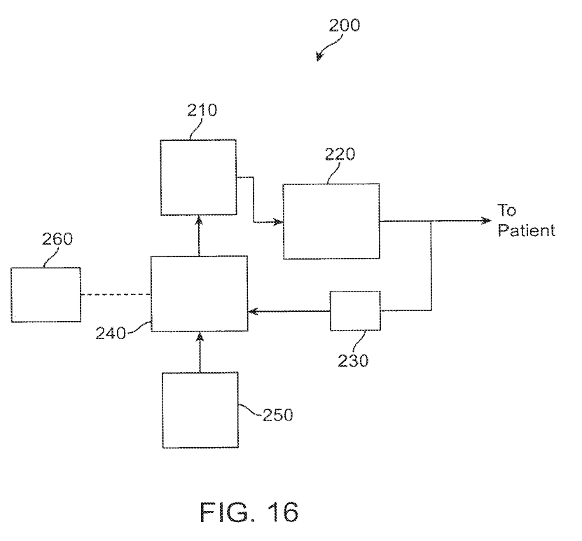

A system according to an embodiment of the present invention is described with reference to FIG. 16. The system 200 can include incorporated therewith any device of the present invention for delivery of energy to the patient, and includes a power unit 210 that delivers energy to a driver unit 220 and then to electrode(s) of an inventive device. The components of the system individually or collectively, or in a combination of components, can comprise an energy source for a system of the invention. A power unit 210 can include any means of generating electrical power used for operating a device of the invention and applying electrical current to a target tissue as described herein. A power unit 210 can include, for example, one or more electrical generators, batteries (e.g., portable battery unit), and the like. One advantage of the systems of the present invention is the low power required for the ablation process. Thus, in one embodiment, a system of the invention can include a portable and/or battery operated device. A feedback unit 230 measures electric field delivery parameters and/or characteristics of the tissue of the target tissue region, measured parameters/characteristics including without limitation current, voltage, impedance, temperature, pH and the like. One or more sensors (e.g., temperature sensor, impedance sensor, thermocouple, etc.) can be included in the system and can be coupled with the device or system and/or separately positioned at or within the patient's tissue. These sensors and/or the feedback unit 230 can he used to monitor or control the delivery of energy to the tissue. The power unit 210 and/or other components of the system can be driven by a control unit 240, which may be coupled with a user interface 250 for input and/or control, for example, from a technician or physician. The control unit 240 and system 200 can be coupled with an imaging system 260 (see above) for locating and/or characterizing the target tissue region and/or location or positioning the device during use,

A control unit can include a, e.g., a computer or a wide variety of proprietary or commercially available computers or systems having one or more processing structures, a personal computer, and the like, with such systems often comprising data processing hardware and/or software configured to implement any one (or combination of) the method steps described herein. Any software will typically include machine readable code of programming instructions embodied in a tangible media such as a memory, a digital or optical recovering media, optical, electrical, or wireless telemetry signals, or the like, and one or more of these structures may also be used to transmit data and information between components of the system in any wide variety of distributed or centralized signal processing architectures.

Components of the system, including the controller, can be used to control the amount of power or electrical energy delivered to the target tissue. Energy may be delivered in a programmed or pre-determined amount or may begin as an initial setting with modifications to the electric field being made during the energy delivery and ablation process. In one embodiment, for example, the system can deliver energy in a “scanning mode”, where electric field parameters, such as applied voltage and frequency, include delivery across a predetermined range. Feedback mechanisms can be used to monitor the electric field delivery in scanning mode and select from the delivery range parameters optimal for ablation of the tissue being targeted.

Methods and techniques of the present invention may employ a single device or a plurality of devices. In one embodiment, for example, a device of the present invention (e.g., device as illustrated in FIGS. 2A through 2C) can be positioned within a target tissue region as described above. A second device can then be positioned within the target tissue region or in another target tissue region, either of part of the same tumor or at a separate tumor. In one embodiment, for example, a first device is positioned in a target tissue region, and a second device can be positioned in the target tissue region, where the second device is positioned at an angle (e.g., 90 degree angle) relative the first device. Additionally, the same device may be positioned in a different orientation and/or location at a separate time point.

Systems and devices of the present invention can, though not necessarily, be used in conjunction with other systems, ablation systems, cancer treatment systems, such as drug delivery, local or systemic delivery, radiology or nuclear medicine systems, and the like. Similarly, devices can be modified to incorporate components and/or aspects of other systems, such as drug delivery systems, including drug delivery needles, electrodes, etc.

In some instances, it may be desirable to remove ablated tissue from the target tissue region at a stage of the ablation process described herein. For example, it has been observed that, in some instances, removal of ablated tissue can improve treatment and/or recovery of the subject, and possibly reduce stress and/or toxicity (e.g., local tissue toxicity, systemic toxicity, etc.) associated with the ablation process of the present invention.

Various devices and methodologies can be utilized for removing the ablated tissue. In some instances, as described above, the ablated tissue can effectively “liquefy” or assume properties of a liquid-like material. The liquid ablated tissue can then be drained or removed from the target tissue region. In one embodiment, removal of the ablated tissue can be as simple as allowing ablated tissue to leak or ooze out of target tissue region (e.g., with or without application of a force or pressure to the target tissue region or tissue proximate thereto), for example, by leaking out holes or piercings in the tissue, including, e.g., entry holes through which the device/electrodes are introduced into the target tissue region. In other embodiments, removal of ablated tissue can be more deliberate or controlled. The removal can be accomplished using a device or apparatus separate from the ablation device, such as a syringe or other liquid removing device, or the removal can be accomplished using the ablation device further configured for the tissue removal.

While some embodiments of the present invention can include positioning of an electrode directly within and at the approximate center of the target tissue region, in some instances it may be desirable to apply an electric field as described above, through the target tissue region, in the absence of an electrode positioned centrally within the defined ablation volume. Referring to FIGS. 17A through 17D, an ablation probe/device of the present invention according to another embodiment of the present invention is described. The device 270, as illustrated in FIG. 17A, is configured for delivery of an electric field to a target tissue region (“T”) such that the electric filed is applied through target tissue region and in a plurality of different directions. The device includes a plurality of electrodes 272 that can be positioned to substantially define an ablation volume or target region. In some embodiments, electrodes can he deployable from a catheter-type device (e.g., similar to as configurations described above), e.g., from a distal portion, that can be advanced to the a target region. Similar to embodiments described above, the device 270 can include a delivery member having a lumen with microcatheters positioned within the lumen of a delivery member, and electrodes 272 each disposed in a microcatheter. As illustrated, microcatheter 273 can be deployed from the delivery member and may act as an initial advancement or guide tube as advanced or deployed from delivery member for initial aiming and/or positioning of electrode disposed therein. In use, treatment can include activation of electrodes 272 (e.g., opposing electrodes) in pairs, such that the electrode pairs define a circuit and an applied field extends between the two electrodes of the pair. Different electrode pairs can be activated to apply electric fields to different portions of the target tissue and/or fields having different directions/orientations. Electrodes can be configured to have defined electrically active areas, for example, by including insulated and non-insulated portions. FIG. 17B illustrates activation of opposing electrode pairs 274, 276 of a device that can include a plurality of electrode pairs, and field generation between the activated electrode pairs as illustrated by the arrows. Electrodes 274, 276 can each include active portions 278, 280, respectively. Electrodes can each include a single or continuous active area, as shown, or a plurality of active areas along a length of an electrode (not shown). Active areas can he positioned at various locations on electrodes so as to select the direction/orientation of the field applied by a given pair (see, e.g., FIG. 17C). FIG. 17D illustrates an embodiment of an electrode pair 282, 284 having an electrode configuration for generating a field that runs approximately parallel to the longitudinal axis of the probe. A device can include a plurality of electrode pairs configured as described, with different pairs of the plurality applying fields in different directions across the target tissue. Configuration and arrangement of electrodes in this manner can permit application of fields through the tumor and in a plurality of different directions. Current can be applied such that fields extend substantially through an approximate central region of the volume, as shown. Though various configurations of electrodes and/or active areas on electrodes can be included in the present invention, including probes/electrodes/active areas configured such that applied fields extend through a central region, or through regions of the volume other than the center, or both. Electrode pairs can be activated individually or sequentially such that only one electrode pair is activated at any one moment, or multiple pairs can be activated simultaneously.

While embodiments of the present invention are discussed in terms of use for non-thermal ablation and destruction of cancerous cells as described above, in some instances systems and probes can be configured for delivering energy sufficient for other types of tissue ablation, such as thermal RF ablation, microwave ablation, irreversible electroporation via high-voltage direct current, and the like. For example, a system of the invention can include a power unit configured for delivery of energy suitable for any one or more types of tissue ablations. In fact, certain probe configurations have designs (e.g., electrode arrangements) that can provide improved delivery of a various types of tissue ablation, including, e.g., improved delivery of thermal RF ablation, and the like. And treatment according to methods of the present invention can include delivery of one or more types of tissue ablations for a given treatment. In some instances, for example, treatment may include one or more ablation delivery modes, such as one mode where non-thermal tissue ablation is delivered, which can precede or follow another ablation mode, such as thermal RF tissue ablation. For example, in one embodiment, treatment can include delivery of non-thermal tissue ablation followed by a shorter application or pulse of energy to produce a thermal mediated effect, e.g., to help “sterilize” one or more components of the probe for withdrawal from the target tissue through the entry track and reduced risk of tracking any potentially viable cancer cells through tissue during probe withdrawal.

In some embodiments, systems of the present invention can further include certain components and aspects for positioning and/or stabilizing probes and other components during the energy delivery process. For example, in instances where a phase of treatment, such as energy application, is expected to exceed more than a few minutes, it may be desirable to include a positioning or stabilizing structure to maintain a probe in a desired position/location without specifically requiring a user surgeon) to hand-hold the probe. Thus a system can include a harness, belt, clamp, or other structure to maintain probe positioning. Systems can be designed for ambulatory use so as to allow for movement of the patient (e.g., shifting, walking, etc.) during treatment. In fact, the low-power requirements and corresponding design options (e.g., battery powered system) may make the current systems particularly well suited for use as an ambulatory system.

It is understood that the examples and embodiments described herein are for illustrative purposes only and that various modifications or changes in light thereof will be suggested to persons skilled in the art and are included within the spirit and purview of this application and scope of the appended claims.

What is claimed is:

1. A method of delivering an electrical field to a tissue, comprising:
advancing a probe comprising a lumen and a plurality of elongate electrodes in a stowed configuration, in which the plurality of elongate electrodes is at least partially disposed within the lumen, to a target tissue region comprising abnormally proliferating cells;
deploying the plurality of elongate electrodes, configured as one or more pairs of elongate electrodes, together from the stowed configuration from a distal end of the lumen so as to penetrate and extend into the tissue and define a treatment volume of the tissue between the deployed elongate electrodes, wherein a portion of each elongate electrode of the one or more pairs of elongate electrodes is positioned and curves inwardly toward a longitudinal axis of the probe to define the treatment volume, the deployed elongate electrodes positioned to define the treatment volume; and
applying an alternating electrical current to the treatment volume so as to provide one or more electric fields extending through the treatment volume at less than 50 V/cm for a treatment time of less than 3 hours, selectively destroying abnormally proliferating cells within the treatment volume compared to normal cells within the treatment volume.

2. The method of claim 1, wherein elongate electrodes of the plurality of elongate electrodes each comprise an insulated portion and a non-insulated portion.

3. The method of claim 2, wherein a non-insulated portion of an elongate electrode defines an electrically active portion of the elongate electrode.

4. The method of claim 1, wherein the plurality of elongate electrodes are activated in pairs, with elongate electrodes of each pair having an opposing polarity.

5. The method of claim 4, wherein elongate electrode pairs are activated in a sequence.

6. The method of claim 1, wherein the destruction of abnormally proliferating cells comprises low-power, and mild hyperthermia comprising an average tissue temperature less than about 48 degrees C.

7. The method of claim 6, wherein the alternating electrical current comprises an alternating electrical current having a frequency between about 50 kHz and about 300 kHz.

8. The method of claim 1, wherein the treatment volume comprises a tumor and the tumor is substantially disposed within the treatment volume.

9. The method of claim 1, wherein an applied voltage field is substantially aligned with division axes of dividing abnormally proliferating cells of the treatment volume.

10. The method of claim 1, wherein the applied one or more electric fields disrupts cellular membrane integrity or cell cycle progression of dividing abnormally proliferating cells.

11. The method of claim 1, wherein the applied one or more electric fields provides at least partial liquification of abnormally proliferating cells of the treatment volume.

12. A method of delivering an electrical field to a tissue, comprising:
advancing a probe comprising a plurality of elongate electrodes, the plurality of elongate electrodes configured as one or more pairs of elongate electrodes, together to a target tissue region comprising abnormally proliferating cells;
positioning the plurality of elongate electrodes so as to penetrate and extend into the tissue and define a treatment volume of the tissue between the positioned plurality of elongate electrodes, wherein a portion of each elongate electrode of the one or more pairs of elongate electrodes are positioned and curves inwardly toward a longitudinal axis of the probe to define the treatment volume; and
preferentially ablating abnormally proliferating cells of the treatment volume compared to normal cells within the treatment volume for a treatment time less than 3 hours, the ablating comprising delivering alternating current to the tissue within the treatment volume so as to establish a plurality of electric fields extending through an approximate center location of the treatment volume less than 50 V/cm in the plurality of fields, the plurality of fields comprising a first electric field, a second electric field having an angle relative to the first field, and a third electric field having an angle relative to the first and second fields.

13. The method of claim 12, the first field extending between active portions of a first pair of opposing elongate electrodes; the second field extending between active portions of a second pair of elongate electrodes; and the third field extending between active portions of a third pair of elongate electrodes.

14. The method of claim 13, wherein the active portions comprise non-insulated portions of the elongate electrodes.

15. The method of claim 1, wherein the abnormally proliferating cells are substantially disposed within the treatment volume.

16. A device for delivering an electric field to a tissue to destroy abnormally proliferating cells therein, the device comprising:
a probe having a plurality of elongate electrodes positionable together at a target tissue region and deployable from a distal portion of the probe so as to penetrate and extend into the tissue and define a treatment volume of the tissue between the deployed elongate electrodes, wherein a portion of each elongate electrode is positioned and curves inwardly toward a longitudinal axis of the probe to define the treatment volume, such that the treatment volume of the tissue is defined by the positioned elongate electrodes,
the plurality of elongate electrodes comprising elongate electrode pairs configured to provide electric fields extending through the treatment volume and preferentially destroying abnormally proliferating cells within the treatment volume compared to normal cells within the treatment volume,
the probe configured for activation of different pairs of elongate electrodes sequentially such that each elongate electrode pair defines a circuit and an applied alternating current field extends between two elongate electrodes of an activated elongate electrode pair, and the sequential activation of different pairs establish electric fields radially and in a plurality of different directions and extending through an approximate central region of the treatment volume, and wherein each elongate electrode pair is configured to deliver an electric field having a different angle relative to fields delivered from a different pair of elongate electrode pairs of the plurality of elongate electrodes; and
wherein the alternating electrical current provides a voltage field less than 50 V/cm in the electric fields for a treatment time less than 3 hours to selectively destroy the abnormally proliferating cells within the treatment volume compared to the normal cells within the treatment volume.

17. The device of claim 16, wherein each elongate electrode of the plurality of elongate electrodes comprises an electrically active portion.

18. The device of claim 17, wherein the electrically active portion comprises a non-insulated portion of the elongate electrode.

19. The device of claim 16, further comprising a microcatheter tube deployable from the distal portion of the probe and an elongate electrode of the plurality of elongate electrodes deployable from the microcatheter tube.

20. The device of claim 16, wherein the plurality of elongate electrodes is positionable such that applied electric fields are substantially aligned with division axes of dividing abnormally proliferating cells in the treatment volume.

21. A system for energy delivery and induction of mild hyperthermia in a target tissue region for preferential ablation of abnormally proliferating cells, comprising:
a probe having a plurality of elongate electrodes positionable together at a target tissue region, the plurality of elongate electrodes configured as one or more pairs of elongate electrodes, and deployable together from a distal portion of the probe so as to penetrate and extend into the tissue and define the target tissue region between the deployed elongate electrodes, wherein a treatment volume is defined by the plurality of deployed elongate electrodes, wherein a portion of each elongate electrode of the one or more pairs of elongate electrodes curves inwardly toward a longitudinal axis of the probe to define the treatment volume when deployed, and provide electric fields extending through the target tissue region;
an energy source coupled to the device to provide an alternating electrical current selected to induce mild hyperthermia comprising an average tissue temperature less than about 48 degrees C., so as to preferentially ablate abnormally proliferating cells of the tissue disposed within the target tissue region compared to normal cells disposed within the target tissue region; and
wherein the alternating electrical current provides a voltage field less than 50 V/cm in the electric fields for a treatment time less than 3 hours to selectively destroy the abnormally proliferating cells within the treatment volume compared to the normal cells within the treatment volume.

22. The system of claim 21, wherein the energy source is powered by a battery.

23. The system of claim 21, wherein the system comprises an electrically floating system.

24. The system of claim 21, further comprising a feedback unit for detecting a characteristic of tissue of the target tissue region, a characteristic comprising impedance and/or temperature and/or pH.

25. The system of claim 21, further comprising a tissue removal system.

26. The system of claim 21, further comprising an imaging system.

27. The system of claim 21, further comprising a computer coupled to the energy source to output a signal for a selected treatment current parameter for application to the target tissue region.

28. The system of claim 27, the selected treatment current parameter comprising current, voltage or frequency.

29. A system for energy delivery and induction of mild hyperthermia in a target tissue region for preferential ablation of abnormally proliferating cells in a target tissue, comprising:
a probe having a plurality of elongate electrodes configured as one or more pairs of elongate electrodes and positionable throughout a target tissue region together so as to penetrate and extend into the tissue and define a treatment volume of the target tissue region between the positioned elongate electrodes, wherein a portion of each electrode of the one or more pairs of electrodes is positioned and curves inwardly toward a longitudinal axis of the probe to define the treatment volume, the elongate electrodes positioned to define the treatment volume of the tissue and provide alternating current (AC) fields extending through the treatment volume in a plurality of different directions when the plurality of elongate electrodes are activated wherein the alternating current provides a voltage field less than 50 V/cm applied in the AC fields for a treatment time less than 3 hours to selectively destroy the abnormally proliferating cells within the treatment volume compared to the normal cells within the treatment volume; and
an energy source coupled to the system to provide an alternating electrical current to the treatment volume, the alternating electrical current selected to induce the AC fields extending through the treatment volume and a mild hyperthermia heating of the treatment volume so as to preferentially ablate abnormally proliferating cells disposed in the treatment volume compared to normal cells within the treatment volume.
